(19) United States
(12) Patent Application Publication
(10) Pub. No.: US 2025/0352503 A1
(43) Pub. Date: Nov. 20, 2025

(54) BIO-OPTIMIZED NUTRACEUTICAL DIETARY SUPPLEMENT

(71) Applicants: Abeer Alghananeem, Louisville, KY (US); Roger Griggs, West Chester, OH (US)

(72) Inventors: Abeer Alghananeem, Louisville, KY (US); Roger Griggs, West Chester, OH (US)

(21) Appl. No.: 18/664,917

(22) Filed: May 15, 2024

(51) Int. Cl. A61K 31/198; A61K 31/12; A61K 31/197; A61K 31/375; A61K 31/4415; A61K 31/51; A61K 31/519; A61K 31/714; A61K 33/06; A61K 36/35; A61K 36/53; A61K 38/48; A61P 25/22

(52) U.S. Cl. CPC A61K 31/198; A61K 31/12; A61K 31/197; A61K 31/375; A61K 31/4415; A61K 31/51; A61K 31/519; A61K 31/714; A61K 33/06; A61K 36/35; A61K 36/53; A61K 38/4873; A61P 25/22

(57) ABSTRACT

A bio-optimized nutraceutical dietary supplement formulation for the management of one or more symptoms associated with intermittent (situational) anxiety, and related disorders may include: Vitamin C, Thiamin, Vitamin B6, Folate, Vitamin B-12, Pantothenic Acid, Magnesium, Curcumin, L-Lysine, Lemon Balm Extract, L-Glycine, Valerian Root, L-Theanine, and Bromelain. The formulation may be formed into capsules for delivery to a user, wherein a therapeutically effective dosage of the formulation may be split between two capsules.

### BACKGROUND OF THE INVENTION

The present invention relates to dietary supplements. and, more particularly, to a bio-optimized dietary supplement for management of one or more symptoms associated with intermittent (situational) stress and anxiety disorders.

Anxiety Disorders affect 18.1% of adults in the United States per National Institute of Mental Health (NIMH), including approximately 6.8 million who suffer from generalized anxiety disorder. Individuals may experience various forms of acute anxiety as well as chronic anxiety episodes, with the most extreme being the panic attack. Panic attacks are a form of extreme anxiety typified by both an exaggerated physiological and psychological response such that the individual may feel as if they are losing control or are similarly overwhelmed. For an individual suffering from a panic attack, the experience can be both frightening and debilitating. For some, panic attacks can be triggered due to a progression of seemingly low stress events but build to a crescendo of extreme anxiety. Currently, a panic attack is a medical situation requiring the intervention of a healthcare professional.

Acute anxiety may be manifest in numerous forms with social phobia (or social anxiety disorder) as an example. Social anxiety disorder is characterized by overwhelming anxiety and excessive self-consciousness in common everyday social situations. Social phobia can be limited to only specific situations-such as a fear of speaking, shyness, and a fear of humiliation. In its most severe circumstances, the individual may experience anxiety just being in the presence of strangers.

People with an anxiety disorder are 3 to 5 times more likely to go to the doctor and 6 times more likely to be hospitalized for psychiatric disorders than those who do not suffer from anxiety disorders. The World Health Organization (WHO) has stated that the direct cost of occupational stress in 1992 in the United States of America (USA) was US$42 billion. The impact of high levels of work-related stress can be seen at personal, organizational, and societal levels. Workers who experience high occupational stress are significantly more likely to make a stress-related work cover claim, use significantly more medical resources and contribute less to an organization through increased absenteeism and lost productivity. Prolonged experience of high levels of stress and not being able to meet workplace demands can lead to ‘burnout’. Workers experiencing greater mental and physical health issues are costly to treat, whether the symptoms are chronic or acute, reducing the worker's ability to return to the workplace. The WHO has claimed that in the USA around US$300 billion is lost in productivity, absenteeism, and staff turnover each year due to high levels of occupational stress.

As can be seen, there is a need for a bio-optimized nutraceutical dietary supplement that can effectively manage episodes of intermittent stress and/or anxiety.

### SUMMARY OF THE INVENTION

In one aspect of the present invention, a nutraceutical dietary supplement formulation for the management of situational anxiety is provided. The formulation can include synergistic ratios of: Vitamin C, Thiamin, Vitamin B6, Folate, Vitamin B-12, Pantothenic Acid, Magnesium, Curcumin, L-Lysine, Lemon Balm Extract, L-Glycine, Valerian Root, L-Theanine and Bromelain.

In another aspect of the present invention, a nutraceutical dietary supplement formulation for the management of situational anxiety is provided. The formulation can include 11.4 mg of Vitamin C, 0.4 mg of Thiamin, 0.4 mg of Vitamin B6, 100 μg of Folate, 0.6 μg of Vitamin B-12, 2 mg of Pantothenic Acid, 64 mg of Magnesium, 200 mg of Curcumin, 199 mg of L-Lysine, 110 mg of Lemon Balm Extract, 64.2 mg of L-Glycine, 91.6 mg of Valerian Root, 73.4 mg of L-Theanine and 34.4 mg of Bromelain.

In another aspect of the present invention, the nutraceutical dietary supplement formation can be provided to a user in pharmaceutically acceptable oral dosage forms such as tablets, hard gelatin capsules, soft gelatin capsules, effervescent granules, gummies, gums, suspensions, and solutions.

These and other features, aspects and advantages of the present invention will become better understood with reference to the following drawings, description, and claims.

### BRIEF DESCRIPTION OF THE DRAWINGS

FIG. 1 is a graphical representation of experimental results obtained following treatment with a bio-optimized nutraceutical according to an embodiment of the present invention; Average GAD-7 score for all participants±Standard Error of the Mean, where the level of statistical significance (P<0.10: (*), P<0.05: *, P<0.01: ** and NC when not calculated) is shown compared to baseline (W00).

FIG. 2 is another graphical representation thereof; Percent change from baseline of GAD-7 score for all participants.

FIG. 3 is another graphical representation thereof; Average R-9 score for all participants±Standard Error of the Mean, where the level of statistical significance (P<0.10: (*), P<0.05: *, P<0.01: ** and NC when not calculated) is shown compared to baseline (W00).

FIG. 4 is another graphical representation thereof; Percent change from baseline of R-9 score for all participants.

FIG. 5 is another graphical representation thereof; Average total GAD score for all participants±Standard Error of the Mean, where the level of statistical significance (P<0.10: (*), P<0.05: *, P<0.01: ** and NC when not calculated) is shown compared to baseline (W00).

FIG. 6 is another graphical representation thereof; Percent change from baseline of total GAD score for all participants.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, one embodiment of the present invention is a bio-optimized nutraceutical dietary supplement composition formulated to be used in subjects exhibiting intermittent (situational) anxiety related to work, environment, or challenging life situations, otherwise known as acute or circumstantial anxiety.

The formulation of the present disclosure may be used as a dietary supplement for management of situational, acute, or circumstantial stress and anxiety. This list of possible constituent elements is intended to be exemplary only, and it is not intended that this list be used to limit the formulation of the present application to just these elements. Persons having ordinary skill in the art relevant to the present disclosure may understand there to be equivalent elements that may be substituted within the present disclosure without changing the essential function or operation of the formulation.

In embodiments, a bio-optimized nutraceutical formulation is an oral composition comprising, per serving, Vitamin C, Thiamin, Vitamin B6, Folate, Vitamin B-12, Pantothenic Acid, Magnesium, Curcumin, L-Lysine, Lemon Balm Extract, L-Glycine, Valerian Root, L-Theanine and Bromelain. A particular formulation may comprise about 10.26 mg (milligrams) to 12.54 mg of Vitamin C, about 0.36 mg to 0.44 mg of Thiamin, about 0.36 mg to 0.44 mg of Vitamin B6, about 90 mcg to 0.11 mg dietary folate equivalent (DFE) of Folate, about 0.54 μg (micrograms) to 0.66 μg of Vitamin B-12, about 1.8 mg to 2.2 mg of Pantothenic Acid, about 57.6 mg to 70.4 mg of Magnesium, about 180 mg to 220 mg of Curcumin, about 179 mg to 219 mg of L-Lysine, about 99 mg to 121 mg of Lemon Balm Extract, about 57.78 mg to 70.62 mg of L-Glycine, about 82.44 mg to 100.76 mg of Valerian Root, about 66.06 mg to 80.74 mg of L-Theanine and about 30.96 mg to 37.84 mg of Bromelain.

In a preferred embodiment, a bio-optimized nutraceutical formulation is an oral composition comprising an amount of Vitamin C (as ascorbic acid) not less than (NLT) 11.4 mg, an amount of Thiamin (as thiamine HCl) NLT 0.4 mg, an amount of Vitamin B6 (as pyridoxine HCl) NLT 0.4 mg, an amount of Folate (as folic acid) NLT 100 μg dietary folate equivalent (DFE), an amount of Vitamin B-12 (as cyanocobalamin) NLT 0.6 μg, an amount of Pantothenic Acid (from d-calcium pantothenate) NLT 2 mg, an amount of Magnesium (as magnesium chloride) NLT 64 mg, an amount of Curcumin as Curcuma Longa (Rhizome) Extract NLT 200 mg, an amount of L-Lysine NLT 199 mg, an amount of Lemon Balm Extract (Leaf & Stem) NLT 110 mg, an amount of L-Glycine NLT 64.2 mg, an amount of Valerian (Root) NLT 91.6 mg, an amount of L-Theanine NLT 73.4 mg, and an amount of Bromelain (2400 Gelatin Digesting Unit per gram [GDU/gm]) NLT 34.4 mg.

In an exemplary embodiment, the oral composition disclosed herein may include one or more of the following weight ratios: a thiamin: Vitamin B6 ratio of about 1:1, with a glycine: lysine ratio of about 1:3 by weight; a Pantothenic Acid: Vitamin C: magnesium: curcumin ratio of about 1:5.7:32:100; and/or a bromelain: theanine: valerian root: lemon balm extract ratio of about 1:2:2.7:3.2

In embodiments, the ingredients can be combined in the appropriate volumes and delivered in, for example, capsules, wherein the effective dosage can be 2 capsules.

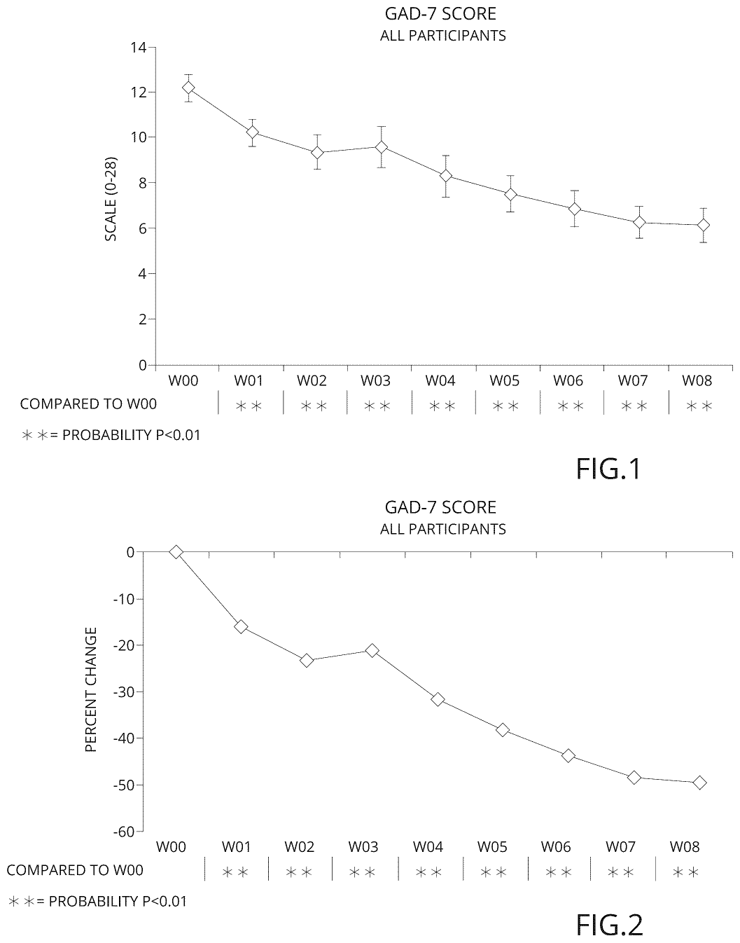

Referring to FIGS. 1-6, results of a clinical evaluation of an embodiment of a bio-optimized nutraceutical formulation are illustrated. Clinical evaluation of a formulation of the present invention was assessed via an open-label study design of 8 weeks' duration (W01-W08). Forty patients were enrolled after a screening process. Patients were selected if they experienced at least three episodes of intermittent, acute, or circumstantial anxiety per week.

Patients consumed the inventive formulation within 30-minutes to one hour prior to, or during, an event or a situation which they anticipated was likely to induce an episode of situational anxiety. Weekly questionnaires were used to collect data over the course of the 8-week study. The questionnaires included Generalized Anxiety Disorder (GAD) Scale composed of GAD-7 module (a validated 7 GAD questions), R-9 module (9 questions developed by the medical advisory team) making a total of 16 GAD (Total GAD) questions. In addition, an Overall Anxiety Severity and Impairment Scale (OASIS) was utilized. The inventive formulation oral composition ratio of thiamin to Vitamin B6 was set as 1:1 by weight, wherein the ratio of the amino acids glycine to lysine was set to around 1:3 by weight. Furthermore, the ratio of Pantothenic Acid to Vitamin C to magnesium to curcumin was set to around 1:5.7:32:100, respectively, by weight. In some embodiments, a weight ratio of bromelain to theanine to valerian root to lemon balm extract is around 1:2:2.7:3.2

Referring to FIGS. 1-2, graphical illustrations of the results of a GAD-7 questionnaire score are illustrated for all participants. Baseline levels were collected at week 0 (W00) and re-evaluations occurred weekly until the conclusion of the trial at week 8. FIG. 1 illustrates Average scores±Standard Error of the Mean and shows a consistent reduction in anxiety commencing immediately at week 1 and continuing through week 8. The level of statistical significance compared to baseline (W00) is indicated as P<0.10: (*), P<0.05: *, P<0.01: **. “Not calculated” is indicated as NC. FIG. 2 illustrates percent change from baseline and shows the same trend as FIG. 1, only scaled as a percent change when each week was compared with week 0. FIGS. 1-2 indicate a long-term benefit associated with regular consumption of the inventive formulation.

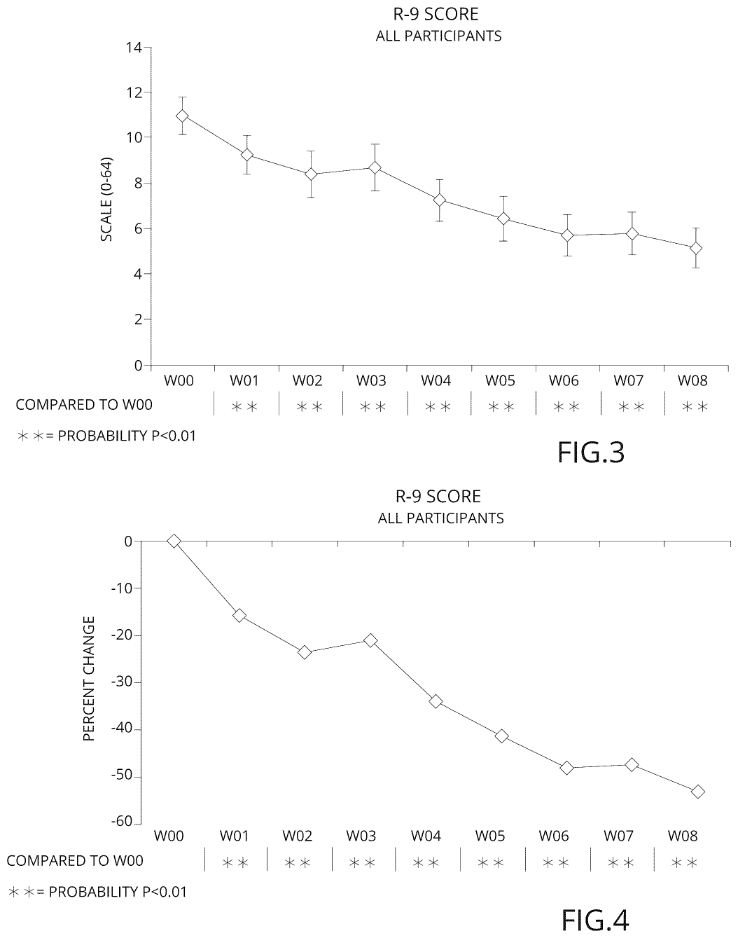

Referring now to FIGS. 3-4, graphical illustrations of the results of R-9 questionnaire scores for all participants are illustrated. Baseline levels were collected at week 0 and re-evaluations occurred weekly until the conclusion of the trial at week 8. FIG. 3 illustrates average scores±Standard Error of the Mean, where the level of statistical significance (P<0.10: (*), P<0.05: *, P<0.01: ** and NC when not calculated) is shown compared to baseline (W00). FIG. 3 demonstrates a consistent reduction in bodily and emotional discomfort commencing immediately at week 1 and continuing through week 8. FIG. 4 illustrates percent change from baseline and shows the same trend as FIG. 1, only scaled as a percentage change when each week was compared with week 0. FIGS. 3-4 indicate a long-term benefit associated with regular consumption of the inventive formulation and corroborate the findings of FIGS. 1-2.

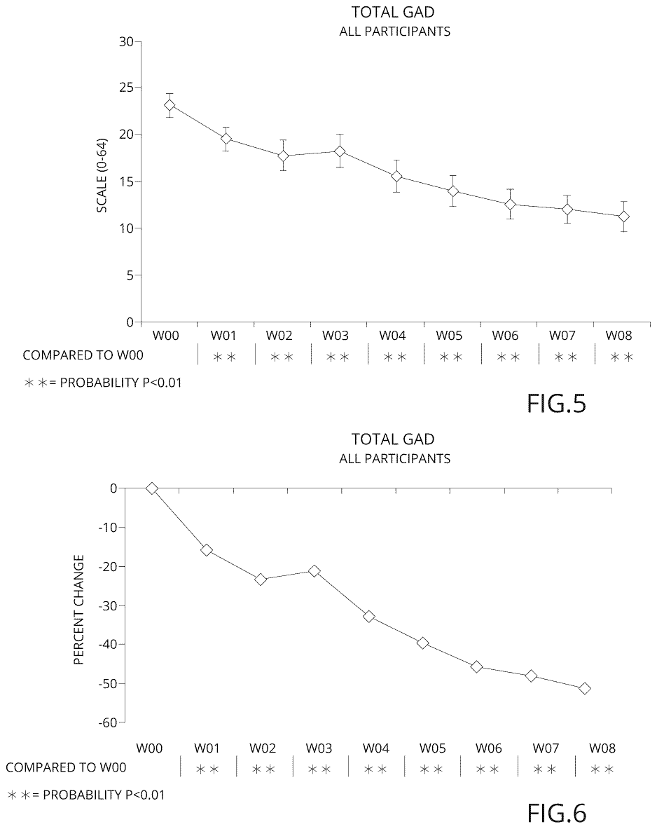

Turning to FIGS. 5-6, graphical illustrations of the results of the OASIS are illustrated as total GAD score for all participants. Baseline levels were collected at week 0 and re-evaluations occurred weekly until the conclusion of the trial at week 8. Data collected using the OASIS showed that the consumption of the dietary supplement as needed over the 8-week study duration was associated with a highly significant reduction in the frequency and severity of anxiety, including a reduced behavioral avoidance from situations causing anxiety, and a reduction in how much anxiety interfered with work, school, social, and family relationships. There were no adverse events during the study that were judged study or product related. FIG. 5 provides average scores±Standard Error of the Mean, where the level of statistical significance (P<0.10: (*), P<0.05: *, P<0.01: ** and NC when not calculated) is shown compared to baseline (W00). FIG. 6 illustrates percent change from baseline.

The trial results confirmed the inventive formulation provided a rapid onset of action which appeared to reduce anxiety after short-term consumption. Furthermore, the data analysis demonstrated broad, sustained, and long-term benefits by lowering the participant's avoidance behavior of anxiety-causing situations with continued consumption of the product. The specific ratios selected, surprisingly, illustrated synergism in the clinical response per the clinical trial outcome.

It should be understood, of course, that the foregoing relates to exemplary embodiments of the invention and that modifications may be made without departing from the spirit and scope of the invention as set forth in the following claims.

What is claimed is:

1. A nutraceutical dietary supplement formulation for management of symptoms associated with situational anxiety, the formulation comprising: Vitamin C, Thiamin, Vitamin B6, Folate, Vitamin B-12, Pantothenic Acid, Magnesium, Curcumin, L-Lysine, Lemon Balm Extract, L-Glycine, Valerian Root, L-Theanine and Bromelain.

2. The nutraceutical dietary supplement formulation of claim 1, wherein a ratio of thiamin:Vitamin B6 by weight is about 1:1.

3. The nutraceutical dietary supplement formulation of claim 1, wherein a weight ratio of Pantothenic Acid:Vitamin C:magnesium:curcumin is about 1:5.7:32:100.

4. The nutraceutical dietary supplement formulation of claim 1, wherein a weight ratio of bromelain:theanine:valerian root:lemon balm extract is about 1:2:2.7:3.2.

5. The nutraceutical dietary supplement formulation of claim 1, wherein a weight ratio of glycine to lysine is about 1:3.

6. The nutraceutical dietary supplement formulation of claim 1, wherein the formulation is provided in a pharmaceutically acceptable oral dosage form selected from the group consisting of: tablets, gelatin capsules, effervescent granules, gummies, gums, suspensions, and solutions.

7. A nutraceutical dietary supplement formulation for management of situational anxiety, the formulation comprising, per serving, not less than (NLT): 11.4 mg of Vitamin C, 0.4 mg of Thiamin, 0.4 mg of Vitamin B6, 100 μg of Folate, 0.6 μg of Vitamin B-12, 2 mg of Pantothenic Acid, 64 mg of Magnesium, 200 mg of Curcumin, 199 mg of L-Lysine, 110 mg of Lemon Balm Extract, 64.2 mg of L-Glycine, 91.6 mg of Valerian Root, 73.4 mg of L-Theanine and 34.4 mg of Bromelain.
